(19) United States
(12) Patent Application Publication
(10) Pub. No.: US 2022/0249661 A1
(43) Pub. Date: Aug. 11, 2022

(54) Pharmaceutical composition for preventing or treating inflammatory bowel disease comprising Tumor necrosis factor alpha inhibitor and Prostaglandin E2

(71) Applicants: SAMSUNG LIFE PUBLIC WELFARE FOUNDATION, Seoul (KR); Research & Business Foundation SUNGKYUNKWAN UNIVERSITY, Suwon (KR)

(72) Inventors: Sung Noh Hong, Seoul (KR); Chansu Lee, Seoul (KR)

(21) Appl. No.: 17/667,885

(22) Filed: Feb. 9, 2022

(30) Foreign Application Priority Data
Feb. 10, 2021 (KR) 10-2021-0019099

(51) Int. Cl. A61K 39/395; A61K 31/5575; A61P 1/12

(52) U.S. Cl. CPC A61K 39/3955; A61P 1/12; A61K 31/5575

(57) ABSTRACT

The present invention relates to a pharmaceutical composition for preventing or treating inflammatory bowel disease containing a tumor necrosis factor alpha inhibitor and prostaglandin E2 that induce regeneration or recovery of damaged intestinal mucosa. As the pharmaceutical composition for preventing or treating inflammatory bowel disease contains the tumor necrosis factor alpha inhibitor and prostaglandin E2 as active ingredients, it may be used to treat inflammatory bowel disease by improving reconstitution of damaged intestinal mucosa and the intestinal mucosal healing ability.

### BACKGROUND

### 1. Technical Field

The present invention relates to a pharmaceutical composition for preventing or treating inflammatory bowel disease containing a tumor necrosis factor alpha inhibitor and prostaglandin E2 that induce regeneration or recovery of damaged intestinal mucosa.

### 2. Related Art

Inflammatory bowel disease is a major digestive disease, and is common in the United States so that it affected 1.33 of all adults (about 3 million people) in 2015. Even in Asia, including Korea, where it is known that there are not many inflammatory bowel disease patients, the number of patients with inflammatory bowel disease has continuously increased. Inflammatory bowel disease can occur in any age group, but it occurs most frequently in young people aged 15 to 35 years. Since inflammatory bowel disease remains uncured during life, it causes pain and large medical expenses, and also causes indirect socioeconomic losses that cannot be estimated, such as loss of labor, by inducing growth disorders and limiting socioeconomic activities. Inflammatory bowel disease is a disease in which abnormal chronic inflammation in the intestinal tract repeats improvement and recurrence. Typical inflammatory bowel diseases include ulcerative colitis and Crohn's disease, and its pathogenesis has not been clearly elucidated. However, taking a look at the studies conducted to date, it is known that chronic inflammation progresses as the mucosal immune system against intestinal microbes is disturbed and boosted due to abnormalities of the barrier function of the intestinal mucosa in patients with a genetic predisposition. Therefore, until now, treatments for inflammatory bowel disease have been developed focusing on treatments for suppressing the immune system of the hyperactive mucosa.

Traditional methods for treating inflammatory bowel disease are performed using 5-ASA, steroids, and immunomodulators to modulate the immune function of the hyperactive intestinal mucosa. However, many patients with inflammatory bowel disease cannot control their symptoms with traditional treatment alone, and thus require a tumor necrosis factor alpha (TNFα) inhibitor which is a more powerful immunosuppressive agent. Tumor necrosis factor is an inflammatory mediator secreted by macrophages, helper T cells, natural killer cells, etc. activated by inflammatory cytokines, and plays a central role in the pathophysiology of inflammatory bowel disease.

TNFα inhibitors bring a breakthrough in the treatment of inflammatory bowel disease and induce symptom improvement in 70 to 80% of patients who do not respond to traditional treatment methods, but with the passage of time, in about ½ of the patients in whom remission was induced for the first time, a loss of response occurs and symptoms recur again. In recent years, various biological agents have been developed, but these drugs show an additional response in only about 40 to 60% of patients.

With the accumulation of experience in treatment with TNFα inhibitors, it has been found that, among patients treated with TNFα inhibitors, the case in which the long-term treatment effect is maintained without loss of response is the case in which mucosal healing is reached in which ulcer of the intestinal mucosa is healed and normal epithelium in the damaged area is regenerated. That is, in the case in which mucosal healing is reached, a stable remission state is maintained for a long time without recurrence. Thus, in recent years, mucosal healing rather than symptom improvement has become a treatment target in inflammatory bowel disease. However, the case in which mucosal healing is achieved with TNFα inhibitors is only 20 to 30% of all inflammatory bowel disease patients, and thus TNFα inhibitors, which are currently considered the best treatment, cannot be presented as a complete solution to reach mucosal healing, which is a treatment target in inflammatory bowel disease. In addition, TNFα inhibitors that are biological agents are expensive and may increase the risk of complications such as the occurrence of infections, skin diseases, tumors, etc.

This reason can be understood by looking at the pharmacological action of TNFα inhibitors. In the mechanism of drug action of TNFα inhibitors, the TNFα inhibitors inhibit TNFα function by neutralizing inflammatory cytokines, inactivate inflammatory signaling pathways induced by TNFα, and induce apoptosis of inflammatory cells that secrete TNFα. Thus, the TNFα inhibitors do not directly promote substantial regeneration of intestinal mucosal epithelial cells. That is, current treatments for inflammatory bowel disease merely target inflammatory substances and immune cells by using TNFα inhibitors and traditional therapeutic agents such as 5-ASA, steroids, and immunotherapeutic agents, but do not target intestinal mucosal epithelial cells. This is thought to be the reason why it is difficult for the current treatments to achieve mucosal healing. That is, although an anti-inflammatory therapeutic agent is effective in the treatment of inflammatory bowel disease, it is insufficient for achieving mucosal healing in most patients, and thus it is important to discover a substance that promotes mucosal healing.

Regeneration and healing of intestinal mucosal epithelium is achieved by regeneration and differentiation of intestinal stem cells. A recent study has reported that intestinal stem cells in organoid derived from intestinal epithelial cells of inflammatory bowel disease patients are different from organoids derived from normal persons. In addition, it has been reported that, in the case of inflammatory patients who received ionizing radiation treatment, intestinal damage is more severe, which indirectly means that intestinal stem cells in inflammatory bowel disease patients are vulnerable to damage. In actual clinical practice, it has been reported that inflammatory bowel disease patients have increased anastomotic leakage after surgery because wound healing is not good. In inflammatory bowel disease, the wound healing ability itself may be damaged due to dysfunction of stem cells. Therefore, it is necessary to evaluate whether the mucosal regeneration ability in patients with inflammatory bowel disease have disorder, and if the regeneration ability is low, a substance capable of improving the mucosal regeneration ability should be discovered and the therapeutic effect thereof alone or in combination with the existing treatment should be evaluated. However, in the past, such research has not been conducted due to methodological limitations of the research. Conducting clinical research is not feasible due to ethical issues because it has to inflict serious harm to actual patients. Laboratory studies can be tried through studies using cell lines and animal studies, but the cell lines are composed of single cells derived from cancer cells with mutations, and thus it is impossible to reproduce the intestinal epithelium composed of multiple cells. In addition, the animal studies have limitations in reproducing the characteristic responses of humans due to biotransformation in non-human animals that do not develop inflammatory bowel disease. The recently established human intestinal organoids consist of all cells constituting the intestinal epithelium, can reproduce the crypt-villus structure, and have the advantage of being able to realize patient characteristics using actual patient-derived cells.

The present invention is based on the results of a study on the use of a pharmaceutical composition containing a tumor necrosis factor alpha inhibitor and prostaglandin E2 for the treatment of inflammatory bowel disease, which has been conducted using patient-derived intestinal organoids and normal person-derived organoids.

### PRIOR ART DOCUMENTS

### Non-Patent Documents

Julian Panes, et al. Crohn's Disease. Drugs. 2007 Vol. 67, No. 17, 2511-2537
Peter Suenaert, M. D., et al. Anti-Tumor Necrosis Factor Treatment Restores the Gut Barrier in Crohn's Disease. The American Journal of Gastroenterology. 2002 Vol. 97, No. 8, 2000-2004
Giulia Roda, et al. Loss of Response to Anti-TNFs: Definition, Epidemiology, and Management. Clinical and Translational Gastroenterology. 2016 Vol. 7, No. 1, e135
Kohei Suzuki, et al. Single cell analysis of Crohn's disease patient-derived small intestinal organoids reveals disease activity-dependent modification of stem cell properties. Journal of Gastroenterology. 2018 Vol. 53, No. 9, 1035-1047
Metcalfe C, et al. Lgr5+ stem cells are indispensable for radiation-induced intestinal regeneration. Cell Stem Cell. 2014 Vol. 14, No. 2, 149-159
Torres J, et al. Crohn's disease. Lancet. 2017 Vol. 389, No. 10080, 1741-1755
Kim J, et al. Human organoids: model systems for human biology and medicine. Nature Reviews Molecular Cell Biology. 2020 Vol. 21, No. 10, 571-584
Sato T, et al. Single Lgr5 stem cells build crypt-villus structures in vitro without a mesenchymal niche. Nature. 2009 Vol. 459, No. 7244, 262-265
Sato T, et al. Long-term expansion of epithelial organoids from human colon, adenoma, adenocarcinoma, and Barrett's epithelium. Gastroenterology. 2011 Vol. 141, No. 5, 1762-1772
Fernandes S R, et al. Proactive Infliximab Drug Monitoring Is Superior to Conventional Management in Inflammatory Bowel Disease. Inflammatory Bowel Diseases. 2020 Vol. 26, No. 2, 263-270
Suzuki K, et al. Single cell analysis of Crohn's disease patient-derived small intestinal organoids reveals disease activity-dependent modification of stem cell properties. Journal of Gastroenterology. 2018 Vol. 53, No. 9, 1035-1047
Khaloian S, et al. Mitochondrial impairment drives intestinal stem cell transition into dysfunctional Paneth cells predicting Crohn's disease recurrence. Gut 2020 Vol. 69, No. 11, 1939-1951

### SUMMARY

An object of the present invention is to provide a pharmaceutical composition for preventing or treating inflammatory bowel disease containing, as active ingredients, a tumor necrosis factor alpha inhibitor and prostaglandin E2 in order to improve the ability to regenerate intestinal epithelium in patients with inflammatory bowel disease.

The present inventors have studied to develop an agent for preventing or treating inflammatory bowel disease, and as a result, have found using intestinal organoids derived from intestinal epithelial cells of Crohn's disease patients that inflammation-inducing TNFα has a negative effect on the regeneration and viability of intestinal epithelial cells in Crohn's disease patients and reduces LGR5-expressing intestinal stem cells, and also have found that treatment with prostaglandin E2 together with a TNFα inhibitor may improve the Crohn's disease treatment effect by improving the intestinal mucosa formation and intestinal mucosa healing ability compared to treatment with the TNFα inhibitor alone, thereby completing the present invention relating to a pharmaceutical composition for preventing or treating inflammatory bowel disease containing a TNFα inhibitor and PGE2.

One aspect of the present invention provides a pharmaceutical composition for preventing or treating inflammatory bowel disease containing a tumor necrosis factor alpha inhibitor and prostaglandin E2 as active ingredients.

As used herein, the term “inflammatory bowel disease” refers to a disease in which abnormal chronic inflammation in the intestinal tract repeats improvement and recurrence, and includes ulcerative colitis and Crohn's disease.

According to one embodiment of the present invention, the inflammatory bowel disease may be Crohn's disease.

As used herein, the term “preventing” refers to any action of inhibiting or delaying the development of inflammatory bowel disease by administering the composition of the present invention, and the term “treating” refers to any action of alleviating or beneficially changing symptoms of inflammatory bowel disease by administering the composition of the present invention.

As used herein, the “tumor necrosis factor” refers to a cytokine that is a cell signaling protein related to inflammatory response and induces an acute phase response. The tumor necrosis factor is classified into called tumor necrosis factor α (TNFα) and tumor necrosis factor β (TNFβ) called lymphotoxin. In the present invention, the term “tumor necrosis factor” and the term “tumor necrosis factor α” may be used interchangeably, and in this case, the term “tumor necrosis factor” means tumor necrosis factor α.

As used herein, the “tumor necrosis factor alpha inhibitor (TNFα inhibitor)” refers to a substance that inhibits the physiological response of tumor necrosis factor alpha and inhibits the activity or function of tumor necrosis factor alpha in the body.

According to one embodiment of the present invention, the tumor necrosis factor alpha inhibitor may be at least one selected from the group consisting of an antibody, a fusion protein and a compound, which specifically bind to tumor necrosis factor alpha.

The antibody may be a monoclonal antibody or polyclonal antibody that recognizes tumor necrosis factor alpha.

According to one embodiment of the present invention, the antibody may be at least one selected from the group consisting of infliximab, adalimumab, certolizumab pegol, golimumab, and etanercept.

The fusion protein is a new protein in which two or more different proteins or homologous proteins are bound to each other. The fusion protein is called a chimera protein, and may be, for example, a tumor necrosis factor-binding receptor produced by artificial manipulation.

The compound may be a naturally occurring or synthesized compound, for example, curcumin, catechins, cannabidiol, or the like.

As used herein, the term “prostaglandin E2 (PGE2)” refers to an active lipid compound produced in various places in the body and having various physiological and pharmacological actions. Prostaglandin E2 generally acts as an inflammatory mediator that promotes local vasodilation and activation of immune cells such as neutrophils, macrophages and mast cells in the early stage of inflammation, as well as promotes the induction of immunosuppressive IL-10 and also limits nonspecific inflammation by inhibiting the production or secretion of several inflammatory cytokines. In the present invention, the composition may contain prostaglandin E2 or a derivative thereof.

The pharmaceutical composition containing the TNFα inhibitor and prostaglandin E2 as active ingredients serves to help reconstitution of the intestinal mucosa damaged due to inflammation or improvement of the intestinal mucosal healing ability.

According to one embodiment of the present invention, the pharmaceutical composition may improve reconstitution of damaged intestinal mucosa and the intestinal mucosal healing ability.

In an example of the present invention, it was confirmed that, when intestinal organoids derived from Crohn's disease patients was treated with TNFα, organoid reconstitution rate, cell proliferation ability and wound healing ability were reduced and cell death increased, compared to those of normal person-derived organoids. This phenomenon is believed to be due to impaired proliferation of LGR5-expressing stem cells in Crohn's disease upon treatment with TNFα. Infliximab, a TNFα inhibitor, inhibits the action of TNFα, thereby inhibiting the occurrence of a response of intestinal organoid cells induced by TNFα. It was shown that the extent to which the cellular response of intestinal epithelial organoids was inhibited by infliximab varied depending on the dose of infliximab. Even in actual clinical practice, there is a difference in the efficacy of infliximab depending on therapeutic drug monitoring of infliximab. Existing studies recommend 3 to 10 μg/mL as the therapeutic blood concentration of infliximab. In the present invention, the concentration of infliximab in medium was set to a low concentration (1 μg/mL) and a high concentration (10 μg/mL), and it was confirmed that the cellular response of intestinal epithelial organoids by TNFα was inhibited and the epithelial regenerative ability was restored. Infliximab at a low concentration (1 μg/mL) in Crohn's disease patient-derived intestinal organoids did not completely restore the TNFα-induced organoid reconstitution rate. In the case of treatment with a high concentration (10 μg/mL) of infliximab, the organoid reconstitution rate, cell proliferation ability and wound healing ability could be restored to normal levels. As expected based on the pharmacological mechanism of action of infliximab, treatment with infliximab did not improve the proliferation of LGR5-expressing stem cells. In actual clinical practice, it may be difficult to maintain the concentration of infliximab at an appropriate therapeutic drug concentration level in many patients. In the present invention, it was confirmed that the incomplete TNFα-induced epithelial cell response by low-concentration infliximab could be restored by promoting the regeneration of intestinal mucosal epithelium in this situation. When the Crohn's disease patient-derived intestinal organoids treated with TNFα were treated with PGE2 in addition to a low concentration (1 μg/mL) of infliximab as a TNFα inhibitor, the organoid reconstitution rate, cell proliferation ability and wound healing ability were improved, similar to when the organoids were treated with a high concentration (10 μg/mL) of infliximab.

Also, according to one embodiment of the present invention, the pharmaceutical composition may induce proliferation of LGR5-expressing intestinal stem cells.

Here, LGR5 is a marker expressed in intestinal stem cells that rapidly differentiate and grow. General stem cells have both self-renewal and differentiation abilities, but do not have the ability to rapidly recycle, and thus LGR5-expressing intestinal stem cells have a high correlation with the regeneration or proliferation of intestinal mucosal cells.

In an example of the present invention, it was confirmed that LGR5-expressing intestinal stem cells increased when normal person-derived intestinal organoids were treated with TNFα, but did not increase in response to TNFα treatment when Crohn's disease patient-derived intestinal organoids were treated with TNFα.

According to one embodiment of the present invention, the pharmaceutical composition may contain prostaglandin E2 and the tumor necrosis factor alpha inhibitor at a weight ratio of 1:100 to 10,000.

More specifically, the weight ratio between prostaglandin E2 and the tumor necrosis factor alpha inhibitor may be 1:100 to 10,000, 1:100 to 8,000, 1:100 to 6,000, 1:100 to 4,000, 1:200 to 10,000, 1:200 to 8,000, 1:200 to 6,000, 1:200 to 4,000, 1:400 to 10,000, 1:400 to 8,000, 1:400 to 6,000, or 1:400 to 4,000.

At this time, if the concentration of the tumor necrosis factor alpha inhibitor is lower than the lower limit of the above range, the inhibition of pathological action of TNFα, improvement in the intestinal mucosa formation and mucosal healing ability, and the effect of proliferating LGR5-expressing intestinal stem cells cannot be expected, and if the concentration of the tumor necrosis factor alpha inhibitor is higher than the upper limit of the above range, there may be no significant difference in the effect, and the cost of treatment may increase, thereby increasing the economic burden on the patient.

In one example of the present invention, it was confirmed that, when TNFα-treated intestinal organoids derived from Crohn's disease patients were treated with 1 μg/mL of infliximab and 5 nM (about 1.75 ng/mL) of PGE2, the organoid reconstitution rate and cell viability were maintained at levels similar to those when the organoids were treated with 10 μg/mL of infliximab or 10 nM (about 3.5 ng/mL) of PGE2 alone.

This pharmaceutical composition contains the TNFα inhibitor and prostaglandin E2 as active ingredients, and is prepared for the purpose of preventing or treating disease. For use, the pharmaceutical composition may be formulated in various forms according to conventional methods. For example, for use, the pharmaceutical composition may be formulated in oral dosage forms such as powders, granules, tablets, capsules, suspensions, emulsions or syrups, and may also be formulated in the form of external preparations, suppositories, or sterile injection solutions. In addition, the pharmaceutical composition may contain a fusion protein of an antibody against TNFα and prostaglandin E2, and may be formulated in the form of external preparations, suppositories, or sterile injection solutions.

The pharmaceutical composition may further include a pharmaceutically acceptable carrier. The carrier is one commonly used in formulation, and examples thereof include, but are not limited to, lactose, dextrose, sucrose, sorbitol, mannitol, starch, gum acacia, calcium phosphate, alginate, gelatin, calcium silicate, microcrystalline cellulose, polyvinyl pyrrolidone, cellulose, water, syrup, methyl cellulose, methylhydroxybenzoate, propylhydroxybenzoate, talc, magnesium stearate and mineral oil. The pharmaceutical composition of the present invention may further contain a lubricant, a wetting agent, a sweetener, a flavoring agent, an emulsifier, a suspending agent, a preservative, and the like, in addition to the above-described ingredients.

In addition, when the pharmaceutical composition is formulated into an ointment, cream, etc., it may be formulated using carriers such as animal oil, vegetable oil, wax, paraffin, starch, tragacanth, a cellulose derivative, polyethylene glycol, silicone, bentonite, silica, talc, zinc oxide, etc.

The dosage of the pharmaceutical composition may vary depending on the formulation method, administration mode, administration time and/or administration route of the pharmaceutical composition. In addition, the dosage of the pharmaceutical composition may vary depending on various factors, including the type and extent of the response to be achieved by administration of the pharmaceutical composition, the type, age, weight, general health status, disease's symptoms or severity, sex, and diet of the subject to whom the pharmaceutical composition is to be administered, excretion rate, drugs used in combination or simultaneously with the composition, and other composition components, as well as similar factors well known in the pharmaceutical field. A person of ordinary skill in the art can easily determine and prescribe an effective dosage for desired treatment. For example, the dosage may be 0.001 to 1,000 mg/kg/day, 0.01 mg/kg/day to 100 mg/kg/day, or 0.1 mg/kg/day to 10 mg/kg/day, but is not limited thereto.

In addition, the pharmaceutical composition may be administered by various routes to mammals such as rats, mice, livestock and humans. The route and mode of administration of the pharmaceutical composition may be independent, and the administration mode is not particularly limited, and any route and mode of administration may be used as long as the pharmaceutical composition can reach the desired site. The pharmaceutical composition may be administered by an oral or parenteral administration method. Examples of the parenteral administration method include, but are not limited to, intravenous administration, intraperitoneal administration, intramuscular administration, transdermal administration, or subcutaneous administration. In addition, a method of applying or spraying the pharmaceutical composition to the diseased site or inhaling the pharmaceutical composition may also be used, but the present invention is not limited thereto.

The pharmaceutical composition for preventing or treating inflammatory bowel disease according to the present invention contains a tumor necrosis factor alpha inhibitor and prostaglandin E2 as active ingredients, and thus may be used to treat inflammatory bowel disease by improving reconstitution of damaged intestinal mucosa and the intestinal mucosal healing ability.

### BRIEF DESCRIPTION OF THE DRAWINGS

FIG. 1 is a schematic view showing a method of establishing normal person- and Crohn's disease patient-derived intestinal organoids according to one example of the present invention.

FIG. 2(A) shows the results of observing subcultured (P1 to P20) normal person- and Crohn's disease patient-derived intestinal organoids with an optical microscope according to one example of the present invention, and FIG. 2(B) shows the results of H&E staining the organoids (scale bar=200 μm).

FIG. 3 is a schematic view showing a process of culturing and differentiating TNFα-treated organoids according to one example of the present invention.

FIG. 4 shows the cell morphology of TNFα-treated normal person- and Crohn's disease patient-derived organoids and the results of analyzing the organoid reconstitution rate of the organoids according to one example of the present invention (scale bar=200 μm).

FIG. 5 shows the results of MTT assay of TNFα-treated normal person- and Crohn's disease patient-derived organoids according to one example of the present invention.

FIG. 6 shows the results of EdU assay of TNFα-treated normal person- and Crohn's disease patient-derived organoids according to one example of the present invention (scale bar=50 μm).

FIG. 7 shows the results of wound healing assay of TNFα-treated normal person- and Crohn's disease patient-derived organoids according to one example of the present invention (scale bar=4 μm).

FIG. 8 shows the results of TUNEL staining of TNFα-treated normal person- and Crohn's disease patient-derived organoids according to one example of the present invention (scale bar=50 μm).

FIG. 9 shows the results of CC3 and TUNEL staining of TNFα-treated normal person- and Crohn's disease patient-derived organoids according to one example of the present invention (scale bar=50 μm).

FIG. 10 shows the results of heatmap analysis (A) and PCA analysis (B) of TNFα-treated normal person- and Crohn's disease patient-derived organoids according to one example of the present invention.

FIG. 11 shows the results of heatmap analysis of epithelial cell-related markers of TNFα-treated normal person- and Crohn's disease patient-derived organoids according to one example of the present invention.

FIG. 12 shows the results of analyzing RPKM values for epithelial cell-related markers of TNFα-treated normal person- and Crohn's disease patient-derived organoids by paired T test (A) and Wilcoxon rank sum test (B) according to one example of the present invention.

FIG. 13 shows the results of analyzing the number of LGR5+ cells of TNFα-treated normal person- and Crohn's disease patient-derived organoids according to one example of the present invention (scale bar=25 μm).

FIG. 14 shows UMAP plots of single-cell RNA sequencing of TNFα-treated normal person- and Crohn's disease patient-derived organoids according to one example of the present invention.

FIG. 15 shows the results of analyzing the RNA expression of epithelial cell-related markers of TNFα-treated normal person- and Crohn's disease patient-derived organoids by single-cell RNA sequencing and heatmap analysis according to one example of the present invention.

FIG. 16 shows the results of analyzing the RNA expression of TNFα signaling-related markers of TNFα-treated normal person- and Crohn's disease patient-derived organoids by single-cell RNA sequencing and heatmap analysis according to one example of the present invention.

FIG. 17 shows cell morphology (a) and the results of organoid reconstitution assay (b) and MTT assay (c) for TNFα-treated normal person-derived organoids following infliximab treatment according to one example of the present invention (scale bar=200 μm).

FIG. 18 shows cell morphology (a) and the results of organoid reconstitution assay (b) and MTT assay (c) for TNFα-treated normal person-derived organoids following PGE2 treatment according to one example of the present invention.

FIG. 19 shows cell morphology (a) and organoid reconstitution assay results (b) for TNFα-treated normal person-derived organoids following infliximab and PGE2 treatment according to one example of the present invention.

FIG. 20 shows cell morphology and organoid reconstitution assay results for TNFα-treated Crohn's disease patient-derived organoids following infliximab and PGE2 treatment according to one example of the present invention.

FIG. 21 shows MTT assay results for TNFα-treated Crohn's disease patient-derived organoids following infliximab and PGE2 treatment according to one example of the present invention.

FIG. 22 shows wound healing assay results for TNFα-treated Crohn's disease patient-derived organoids following infliximab and PGE2 treatment according to one example of the present invention.

FIG. 23 shows the results of qPCR for LGR5 in TNFα-treated Crohn's disease patient-derived organoids following infliximab and PGE2 treatment according to one example of the present invention.

FIG. 24 shows the results of FACS analysis for LGR5 in TNFα-treated Crohn's disease patient-derived organoids following infliximab and PGE2 treatment according to one example of the present invention.

### DETAILED DESCRIPTION

Hereinafter, the present invention will be described in more detail. However, these descriptions are provided for illustrative purposes only to help understand the present invention, and the scope of the present invention is not limited by these illustrative descriptions.

### Experimental Example 1. Intestinal Organoid Formation

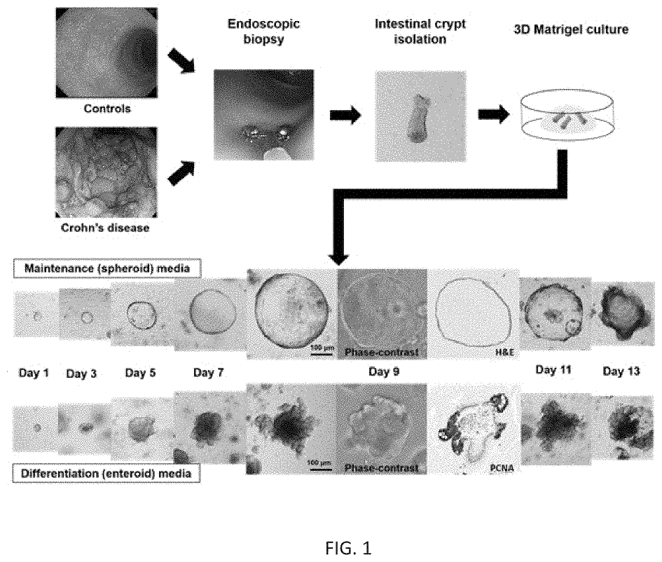

Intestinal mucosal cells were collected from normal persons and Crohn's disease patients and used to establish intestinal organoids (see FIG. 1).

1-1. Sample Collection

To establish intestinal organoids, human intestinal tissues were obtained from normal persons with no inflammatory bowel disease and patients with Crohn's disease by using biopsy forceps during single-balloon enteroscopy at the Samsung Medical Center, Seoul, Korea, between November 2016 and December 2018. At least four biopsy samples were obtained from normal mucosal tissues in the jejunum (100 to 150 cm distal of the ligamentum of Treitz) and ileum. Patients with Crohn's disease were diagnosed according to the guidelines. In patients with Crohn's disease, biopsies were performed at least 5 cm away from the ulcers. This study was approved by the institutional ethical committee of the Samsung Medical Center (IRB No. 2016-02-022), and all biopsy samples were taken with informed consent.

1-2. Intestinal Crypt Isolation

Intestinal crypt isolation from endoscopic biopsy samples was performed with reference to the existing literature (Lei N Y, et al. PLoS One. 2014; 9(1); Lahar N, et al. PLoS One. 2011; 6(11); Khalil H A, et al. PLoS One. 2019; 14(5)). Endoscopic biopsy samples were added to PBS containing 10 mM EDTA (Thermo Fischer) and 1 mM DTT (Thermo Fischer) and incubated on a rocker at 4° C. and 50 rpm for 30 min. The supernatant containing villi and debris was removed. Intestinal crypts were added to fresh PBS and vortexed for sec for duodenum and jejunum samples, 60 seconds for ileum samples, and 120 seconds for colon samples. The supernatant containing crypts was collected and filtered through 70-μm cell strainers (Corning). This process was repeated three times. Combined fractions were centrifuged at 200×g at 4° C. for 2 min, and the pellet was suspended in basal medium (DMEM/F12 (Thermo Fisher) containing antibiotic-antimycotic solution (Thermo Fisher), HEPES (Thermo Fisher) 10 mmol/L, GlutaMAX (Thermo Fisher), 1XN2 (Thermo Fisher), 1XB27 (Thermo Fisher) and N-acetylcysteine (Sigma-Aldrich) 1 mmol/L). The suspension was centrifuged at 200×g at 4° C. for 2 min, and then the pellet was suspended in basal medium.

1-3. Three-Dimensional (3D) Intestinal Crypt Culture

3D intestinal crypt culture was performed with reference to the existing literature (Lei N Y, et al. PLoS One. 2014; 9(1); Lahar N, et al. PLoS One. 2011; 6(11); Khalil H A, et al. PLoS One. 2019; 14(5)). Isolated crypts were pelleted with 3 spins, resuspended in Matrigel (Corning) kept cold on ice, and plated in a dome shape on 48-well culture plates (Corning) heated in an incubator at 37° C. After incubation in the incubator at 37° C. for 15 min, 250 μL of maintenance medium (50% Wnt3a-conditioned medium (ATCC #CRL-2647) and 50% 2× basal medium, supplemented with recombinant human epidermal growth factor (Sigma-Aldrich) 50 ng/mL, recombinant human noggin (R&D Systems) 100 ng/mL, recombinant human R-spondin 1 (PeproTech) 500 ng/mL, 10 mM nicotinamide (Sigma-Aldrich), 10 μM p160ROCK inhibitor (Y27632, selleckchem), 10 μM p38 MAP kinase inhibitor (SB202190, Sigma-Aldrich) and 10 nM Prostaglandin E2 (Cayman Chemical)) was added during the first 2 days. 2.5 μM GSK3 inhibitor (CHIR99021, Stemgent) was added only during the first 2 days.

1-4. Organoid Subculture and Maintenance

After 7 days of culture, the organoids contained in Matrigel were mechanically disrupted by pipetting. The isolated organoids were washed with 10 mL of basal medium and centrifuged at 200×g at 4° C. for 30 sec. The pellet was resuspended in 2 mL of cell dissociation buffer (Thermo Fisher) and incubated in a water bath at 37° C. for 5 min. The cell pellet was resuspended in Matrigel and dispensed in a 48-well culture plate (Corning). After incubation at 37° C. for 15 min, 250 μL of maintenance medium was added. The medium was changed once every two days, and the organoids were passaged at a ratio of 1:2 to 1:4 between 7 and 12 days of culture.

1-5. Intestinal Crypt Differentiation

Organoids can be classified into spheroids and enteroids depending on their shape. Spheroids are defined as round- or oval-shaped organoids with a thin wall composed of a single layer of undifferentiated cells. Enteroids are defined as organoids which have buddings formed along their basolateral side and consist of all components of intestinal epithelial cells including differentiated cells and undifferentiated cells. After 4 to 6 passages, most of the organoids in the maintenance medium formed uniform spheroids and could be passaged stably for a long time. In order to differentiate spheroids into enteroids, the spheroids were cultured in the differentiation medium (maintenance medium without Wnt3A conditional medium, SB202190, nicotinamide and PGE2). The differentiation medium was changed once every 2 days, and enteroids were cultured for 7 to 12 days after passage and then used in the experiment (see FIG. 1).

The cultured organoids were observed using H&E staining or an optical microscope.

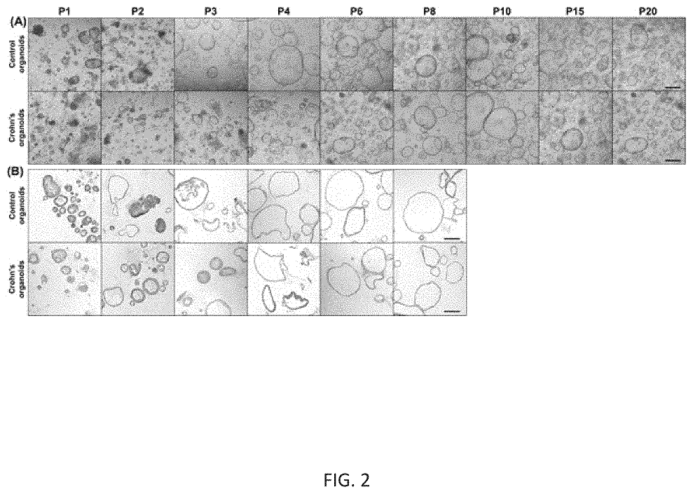

As a result, referring to FIG. 2, it was confirmed that all the organoids derived from normal persons and Crohn's disease patients could be subcultured for 20 passages. In particular, it was confirmed that the organoids derived from Crohn's disease patients did not appear uniform in morphology and did not grow well compared to the normal person-derived organoids in passages 1 to 5 (P1 to P5), but had no difference in cell morphology from the normal person-derived organoids after passage 6 (P6 to P20).

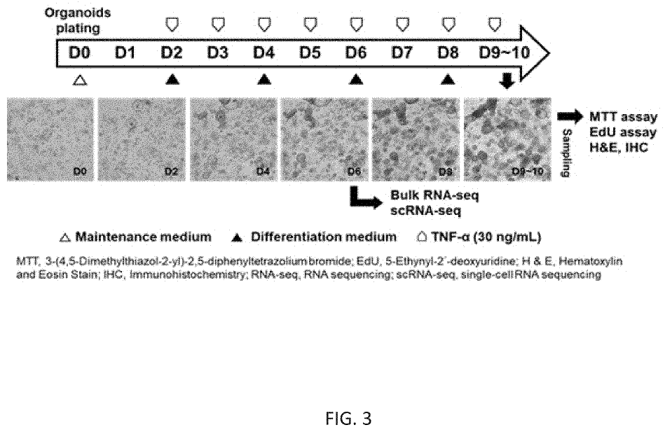

Subsequent experiments were performed using organoids derived from 5 normal persons without inflammatory bowel disease (patients without inflammatory bowel disease or intestinal inflammation) and organoids derived from 5 Crohn's disease patients. Referring to FIG. 3, on day 0 (DO) of subculture of the organoids derived from patients or normal persons, an increase in stem cells and undifferentiated cells was induced by culturing using maintenance medium, and from D2, differentiation of undifferentiated cells was induced by culturing using differentiation medium every other day. 30 ng/mL of TNFα was supplied daily from D2 in order to evaluate the effect of TNFα. After culture on D9 and D10, organoid reconstitution assay, MTT assay, EdU assay, and histological experiments were performed. It was thought that changes in intracellular mRNA would occur 2 to 4 days before the morphological change of the cells was evident. Thus, RNA was extracted from the D6 organoids and subjected to RNA sequencing, and single-cell RNA sequencing was performed.

### Experimental Example 2. Evaluation of Mucosal Regeneration Ability Using Intestinal Organoids

2-1. Organoid Reconstitution Assay

In order to evaluate the organoid-forming ability of intestinal crypts obtained from normal persons and patients with Crohn's disease, 100 intestinal crypts per 25 μL of Matrigel were dispensed into maintenance medium. The culture medium was changed once every two days. The number of viable organoids and enteroids per well was counted under a microscope (CK40, Olympus). The organoid-forming efficiency was calculated as the ratio of available organoids per 100 intestinal crypts. The organoid reconstitution rate was calculated by counting the number of organoids on day 4 and the number of organoids on day 10. Statistical analysis was performed by ANOVA using Bonferroni's multiple comparison test (* p<0.05, ** p<0.01 and *** p<0.001).

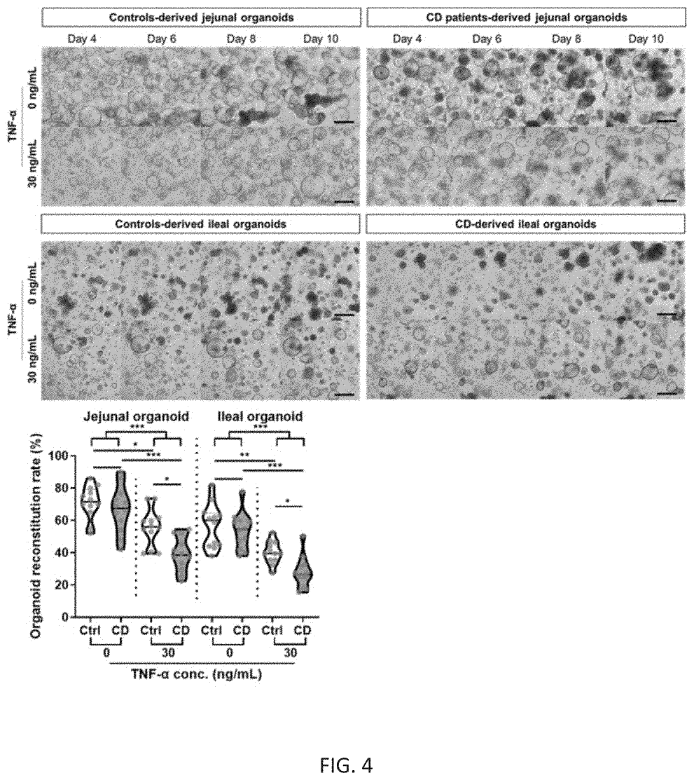

As a result, referring to FIG. 4, it was confirmed that, in the normal person- and Crohn's disease patient-derived organoids cultured in differentiation medium for 10 days, the organoid reconstitution rate decreased when the organoids were treated with TNFα compared to when the organoids were not treated with TNFα. It was confirmed that, when the organoids were not treated with TNFα, there was no difference in organoid reconstitution rates in both the jejunum and ileum between the normal persons and the Crohn's disease patients, whereas when the organoids were treated with TNFα, the reconstitution rates of the Crohn's disease patient-derived organoids in both the jejunum and ileum decreased compared to those of the normal person-derived organoids.

2-2. MTT Assay

To evaluate the cell viability of organoids, 100 μL of basal medium containing MTT (3-[4,5-dimethylthiazol-2-yl]-2,5-diphenyltetrazolium bromide) (Sigma-Aldrich) at a concentration of 500 μg/ml was added to each well of a culture plate and incubated for 2 hours at 37° C. To remove the medium and dissolve the Matrigel, 10 μL of 21 SDS (sodium dodecyl sulfate) solution was added thereto, followed by incubation for 2 hours. After adding 100 μL of detergent reagent, the organoids were further incubated for 1 hour, and the absorbance was measured at 562 nm for quantification. Statistical analysis was performed by ANOVA using Bonferroni's multiple comparison test (* p<0.05, ** p<0.01 and *** p<0.001).

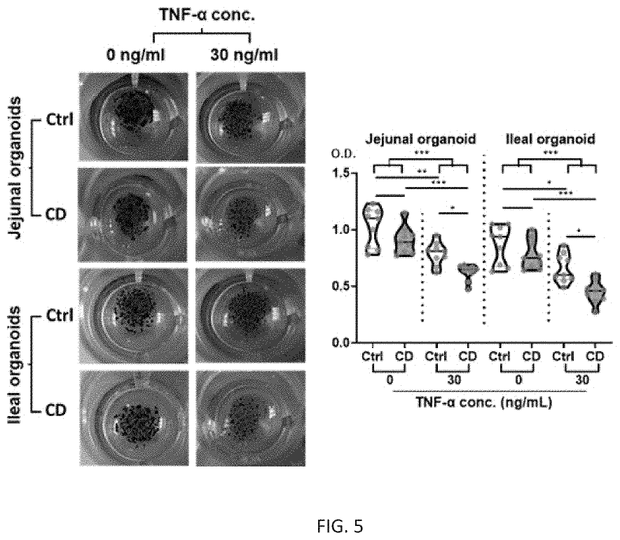

As a result, referring to FIG. 5, it was confirmed that, the cell viability in the TNFα-treated organoids decreased in both the jejunum and the ileum compared to that in the TNFα-untreated organoids.

2-3. EdU Assay

To evaluate the cell proliferation of organoids, each organoid was incubated for 2 hours in a medium containing 10 μM of EdU (5-ethynyl-2′-deoxyuridine) (Abcam), and then fixed with cold 41 paraformaldehyde (Biosesang). EDU incorporation in DNA was detected using Click-iT™ EdU Alexa Fluor® 488 imaging kit (Thermo Fisher). The number of EDU positive (+) cells in the organoids was counted using a confocal microscope (LSM 800, Carl Zeiss). Statistical analysis was performed by ANOVA using Bonferroni's multiple comparison test (* p<0.05 and *** p<0.001).

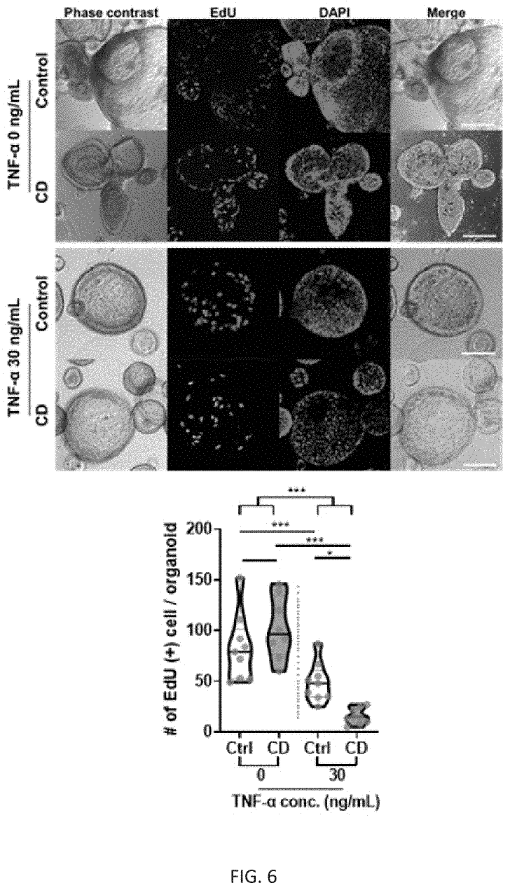

As a result, referring to FIG. 6, it was confirmed that EDU+ cells were located in the buds of the organoids, which mean intestinal crypts, and the number of EDU+ cells in the TNFα-untreated organoids increased compared to that in TNFα-treated organoids. It was confirmed that, when the organoids were not treated with TNFα, there was no difference in the number of EdU+ cells between the normal person-derived organoids and the Crohn's disease patient-derived organoids, whereas when the organoids were treated with TNFα, the reconstitution rate of the Crohn's disease patient-derived organoids decreased compared to that of the normal person-derived organoids.

2-4. Two-Dimensional Human Intestinal Organoid Culture and Wound Healing Assay

For matrix coating, each well of the 24-well plate was coated at 37° C. for 1 hour with 500 μL of Matrigel diluted at a concentration of 51 in the basal medium. For two-dimensional culture, organoids in the three-dimensional culture were digested into single cells using TrypLE Express (Thermo Fisher). The digested cells were resuspended in the basal medium and seeded into Matrigel-coated wells.

For wound healing assay, 5×10⁴ cells were seeded in wells of a 24-well plate containing inserts of CytoSelect™ 24-Well Wound Healing Assay. The two-dimensional organoid monolayers were cultured in maintenance medium until confluence was reached. The inserts were then carefully removed to produce 0.9-mm-diameter wounds, and 500 μL of fresh differentiation medium was added to each well. Then, the cells in the wound were monitored using a phase-contrast microscope at 0, 4, 8, 16, and 24 hours. At each time point, the area of the non-healed wound was measured in 3 different regions. Statistical analysis was performed by ANOVA using Bonferroni's multiple comparison test (* p<0.05, ** p<0.01 and *** p<0.001).

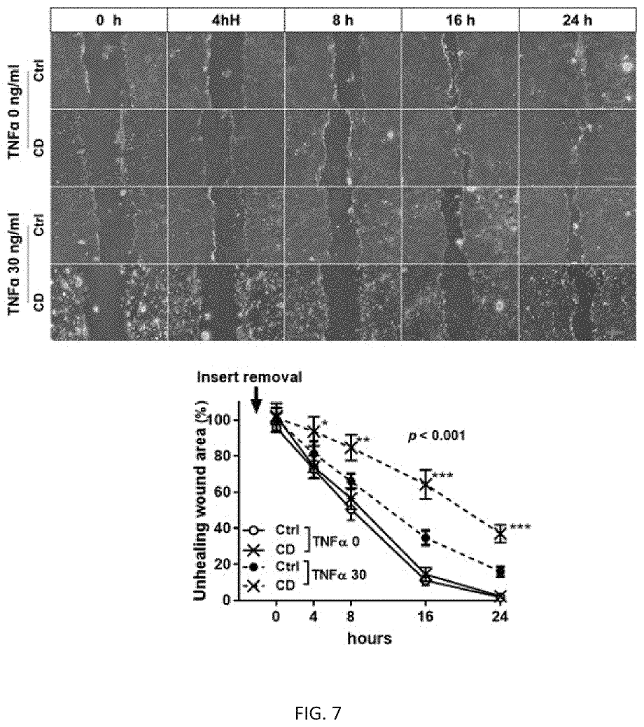

As a result, referring to FIG. 7, it was confirmed that, in the case of the normal person-derived organoids treated with TNFα, the wound area was recovered after 16 hours, indicating that the unhealed wound area was reduced. On the other hand, it could be confirmed that, in the case of the Crohn's disease patient-derived organoids treated with TNFα, there was no significant decrease in the unhealed wound area even over time, indicating that the wound healing ability was reduced.

2-5. Double Immunofluorescence for CC3 and TUNEL

Cell death by TNFα occurs through apoptosis or necroptosis. CC3 (cleaved caspase-3) is a marker capable of detecting cells in which apoptosis has occurred, and TUNEL staining can detect DNA fragmented due to apoptosis, and thus detect cells in which apoptosis or necroptosis has occurred. Therefore, CC3+ and TUNEL+ cells are cells in which apoptosis has occurred, and CC3− and TUNEL− cells are cells in which necroptosis has occurred.

CC3 staining was performed using primary rabbit antibodies specific for CC3 and a goat anti-rabbit secondary antibody conjugated to Alexa 594 fluorochrome (red fluorescence). In TUNEL staining, In Situ Cell Death Detection Kit (Merck) was used for the same sections in CC3 staining, and then fluorescein-labeled dUTP (green fluorescence) was used. DAPI was used to stain intracellular DNA. Statistical analysis was performed by ANOVA using Bonferroni's multiple comparison test (*** p<0.001).

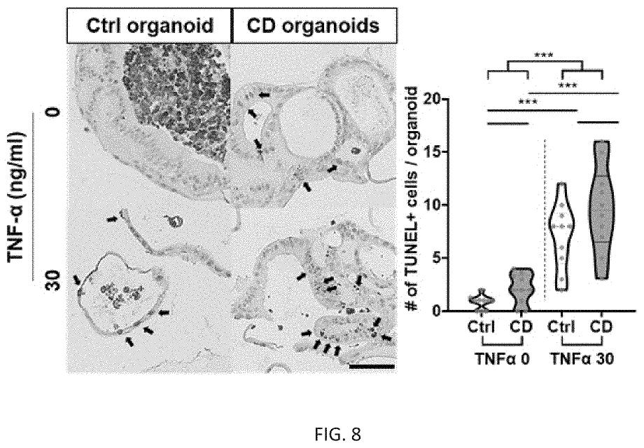

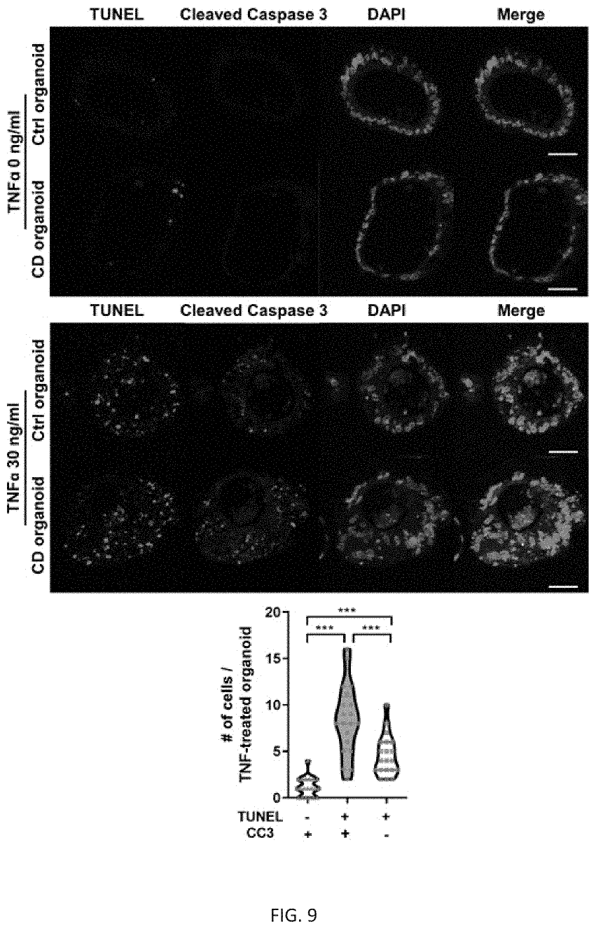

As a result, referring to FIGS. 8 and 9, CC3+ or TUNEL+ cells were hardly observed in the TNFα-untreated organoids. On the other hand, it was confirmed that, in the TNFα-treated organoids, the number of CC3−, TUNEL+ and CC3+, and TUNEL+ cells increased, and in particular, the number of the cells was large in the Crohn's disease patient-derived organoids, indicating that necroptosis more actively occurred than that in the normal person-derived organoids.

These results suggest that TNFα has a negative effect on the intestinal epithelial cell regeneration ability and survival in Crohn's disease patients.

### Experimental Example 3. Evaluation of Cell Function Abnormalities Using Intestinal Organoids

3-1. RNA Sequencing and Data Analysis

Each of normal person-derived and Crohn's disease patient-derived organoids was divided into a TNFα-treated group (n=3) and a TNFα-untreated group (n=3), and cultured in differentiation medium. On day 6 after culture, RNA was extracted from each organoid and subjected to RNA sequencing, and heatmap assay and PCA assay were performed.

RNA sequencing was performed using total RNA samples with >10 μg of RNA and an RNA integrity number (RIN)>8. Libraries for whole transcriptome sequencing were constructed using TruSeq RNA Sample Preparation Kit v2 (Illumina). Reverse transcription reaction was performed with 2 μg of isolated total RNA, poly(dT) primer, and SuperScript™ II Reverse Transcriptase (Invitrogen or Life Technologies). Briefly, RNA sequencing libraries were prepared through complementary DNA amplification, end repair, 3′ end adenylation, adapter ligation and amplification. Library quality and concentration were measured using BioAnalyzer and Qubit systems (Agilent), respectively. The transcriptome libraries were sequenced with the TruSeq Rapid PE Cluster Kit and the TruSeq Rapid SBS Kit (Illumina) using the 100-bp paired-end mode.

Reads from files in the FASTQ format were mapped to the hg19 human reference genome using Tophat version 2.0.6, with default parameters (http://tophat.cbcb.umd.edu/). Raw read counts mapped to genes were measured by using the BAM format file in HTSeq version 0.6.0 (https/htseq.readthedocs.io/) to quantify transcript abundance. The coding genes were selected, and raw read counts were normalized to RPKM (Reads Per Kilobase of transcript, per Million mapped reads) and TMM (Trimmed Mean of M-values). Differential expression analysis of the RNA sequencing experiment was performed with EdgeR (version 3.28.1). Genes differentially expressed in response to TNFα were selected using a paired t-test with FDR (false discovery rate) correction.

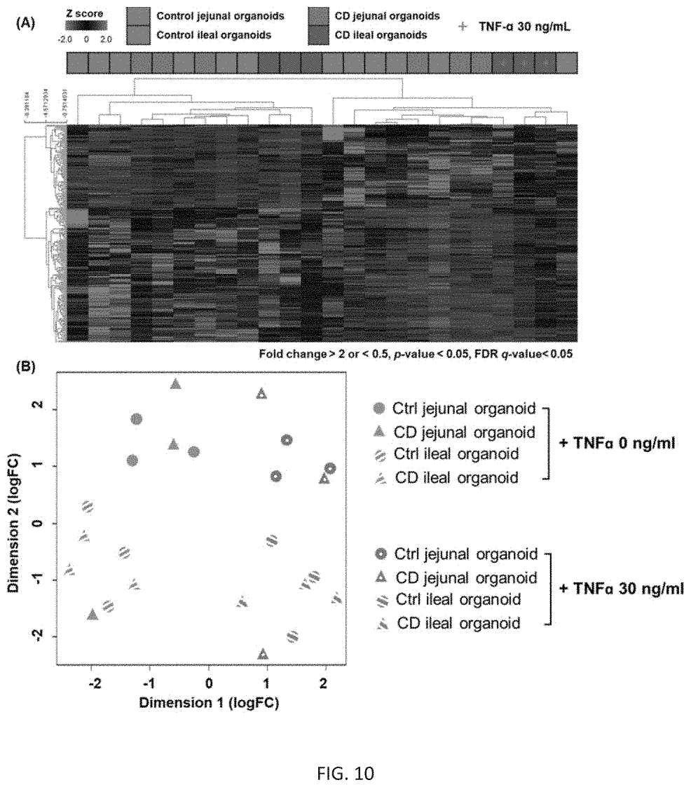

As a result, referring to FIG. 10, it was confirmed that there was a difference in gene expression depending on whether the organoids were treated with TNFα, regardless of the normal person- and Crohn's disease patient-derived organoids.

In addition, using the expression of an epithelial cell lineage-specific marker, the difference between the normal person- and Crohn's disease patient-derived organoids depending on whether or not the organoids were treated with TNFα was analyzed.

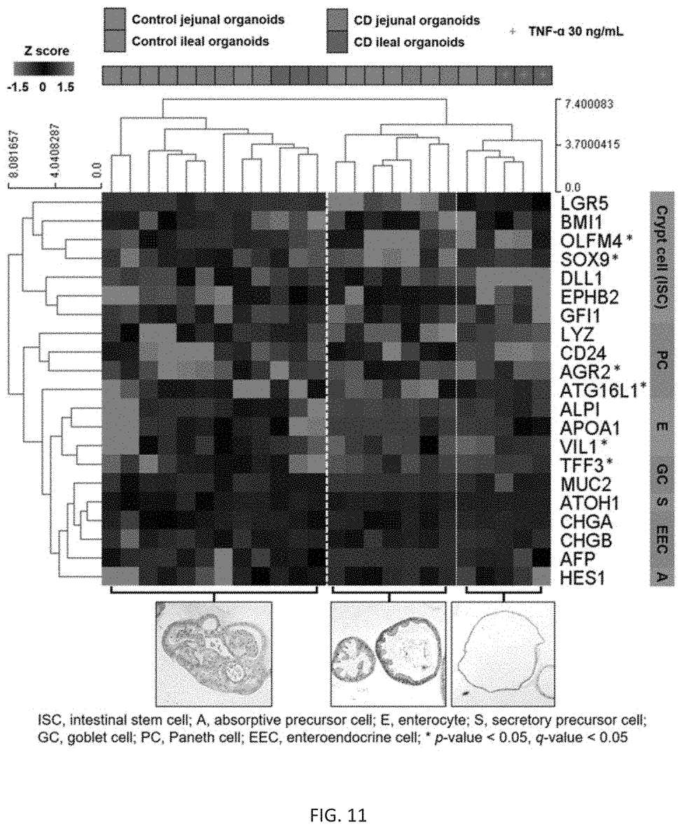

As a result, referring to FIG. 11, it was confirmed that, in the organoids treated with TNFα, the expression of markers of differentiated cells such as enterocytes, goblet cells, and Paneth cells decreased and the expression of markers of intestinal stem cells increased. In particular, in the Crohn's disease patient-derived organoids, a decrease in LGR5 among intestinal stem cell markers was more pronounced.

Changes in RPKM values in the normal person- and Crohn's disease patient-derived organoids depending on whether or not the organoids were treated with TNFα were analyzed.

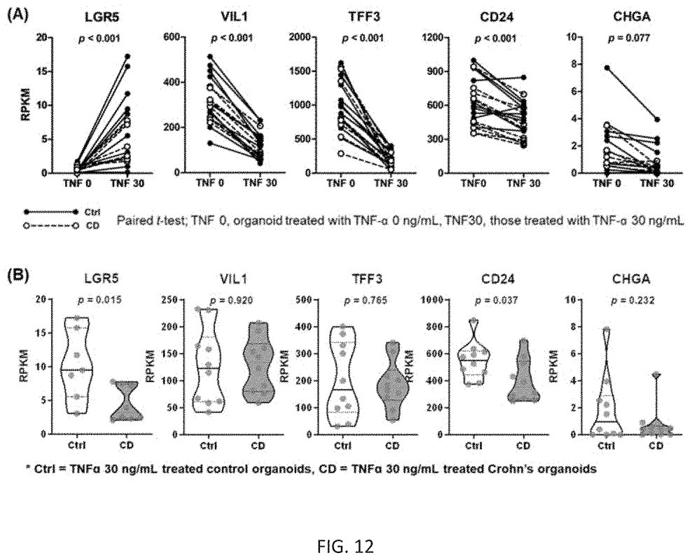

As a result, referring to FIG. 12 (a), it was confirmed that, when the change in RPKM value was statistically analyzed by paired T test, the expression of intestinal stem cell markers such as LGR5 increased in the TNFα-treated organoids compared to the TNFα-untreated organoids, whereas the expression of the enterocyte marker (VIL1), the goblet cell marker (TFF3) and the Paneth cell marker (CD24) decreased.

Referring to FIG. 12(b), it was confirmed that, when the change in RPKM value was statistically analyzed by Wilcoxon rank sum test, the expression of LGR5 and CD24 significantly decreased in the Crohn's disease patient-derived organoids, whereas there was no difference in the expression of VIL1, TFF3 and CHGA.

3-2. Immunohistochemistry (IHC)

After removing the culture medium, the organoids were washed with PBS and fixed in cold 4% paraformaldehyde at room temperature for 30 minutes. The fixed organoids were washed with PBS and embedded in HistoGel (Thermo Fisher). HistoGel blocks were used for paraffin embedding and sectioned for histologic examination and IHC. Histologic evaluation was performed using hematoxylin-eosin-stained sections. After heating-induced epitope retrieval with citrate buffer, IHC was performed using the following antibodies: cleaved caspase-3 (Asp175, 1:200 dilution, Cell Signaling), E-cadherin (1:100 dilution, Abcam), and LGR5 (1:400 dilution, Abcam). The number of LGR5+ cells per organoid in each of the TNFα-treated group (n=10) and the TNFα-untreated group (n=10) was counted, and statistical analysis was performed by ANOVA using Bonferroni's multiple comparison test (*** p<0.001).

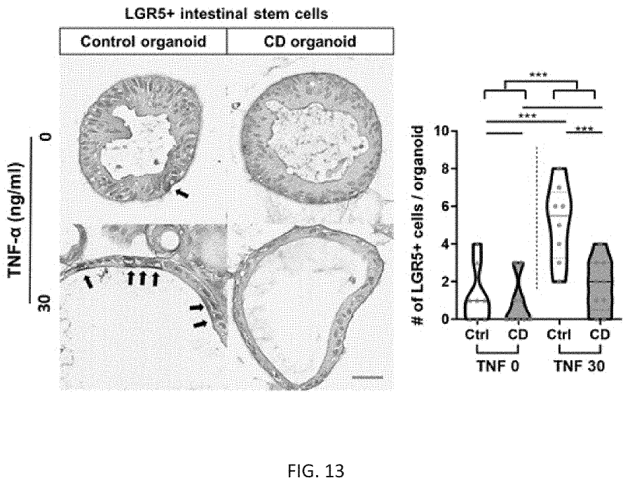

As a result, referring to FIG. 13, it was confirmed that the number of LGR5+ cells increased in the TNFα-treated organoids compared to the TNFα-untreated organoids. However, it was confirmed that, when the organoids were treated with TNFα, the number of LGR5+ cells decreased in the Crohn's disease patient-derived organoids compared to the normal person-derived organoids.

3-3. Single-Cell RNA Sequencing and Analysis

Each of the normal person- and Crohn's disease patient-derived organoids was divided into a TNFα-treated group (n=1) and a TNFα-untreated group (n=1) and cultured in a differentiation medium. On day 6 after culture, the organoids were digested into single cells, and RNA was extracted from each organoid and subjected to RNA sequencing. Differentially expressed genes were divided into 14 clusters, and then a UMAP plot (Uniform Manifold Approximation and Projection plot) was prepared.

Briefly, 10× chromium libraries were prepared according to the manufacturer's protocol (10× Genomics, Flugenton) for 10,000 cells. Barcoded sequencing libraries were generated using Chromium Single Cell 3′ Reagent Kit v3 according to the manufacturer's instructions and sequenced on the HiSeq X Ten system. Reads were aligned to the human reference genome (CGCh38) and processed using the CellRanger 3.1.0 pipeline (10× Genomics). The raw gene expression matrix obtained from the CellRanger pipeline was normalized using Seurat v3.1.4 and filtered according to the following criteria: genes >200, genes <6,000, and <30% of mitochondrial gene expression in UMI counts. The gene expression matrix in the filtered cells was normalized to the total number of UMIs per cell and converted to a natural log scale. In order to combine independent samples, ‘FindIntegrationAnchors’ and ‘IntegrateData’ were used in Seurat and the batch effect was corrected. The number of principal components was estimated using ‘RunPCA’ followed by ‘ElbowPlot’, and dimensionality reduction was performed using ‘RunUMAP’.

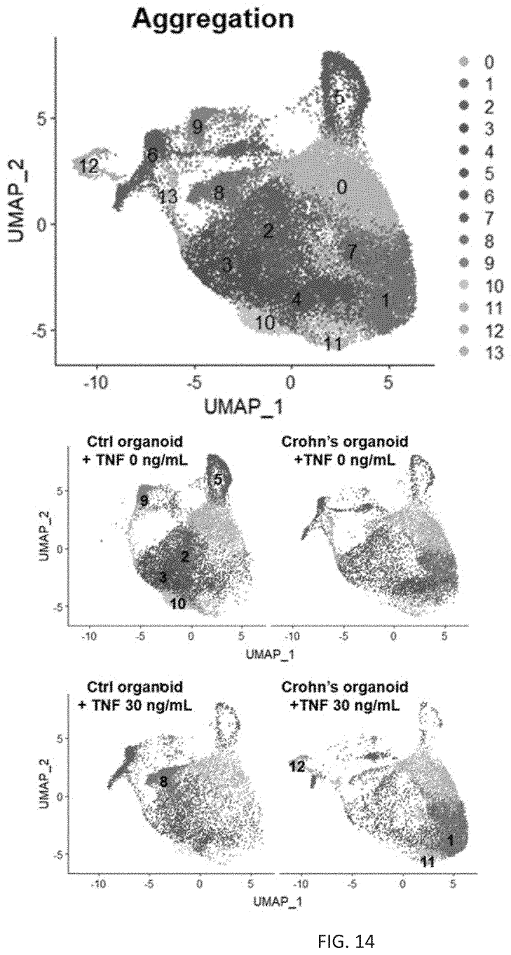

As a result, referring to FIG. 14, it was confirmed that, in the normal person-derived organoids, clusters 2, 3, 5, 9, and 10 were specifically expressed when the organoids were not treated with TNFα, whereas cluster 8 was specifically expressed when the organoids were treated with TNFα. On the other hand, it was confirmed that, in the Crohn's disease patient-derived organoids, clusters 1, 11, and 12 were specifically expressed when the organoids were treated with TNFα.

In addition, using the expression of an epithelial cell lineage-specific marker, the average RNA expression difference between the normal person-derived organoids and the Crohn's disease patient-derived organoids depending on whether or not the organoids were treated with TNFα was analyzed by a heatmap.

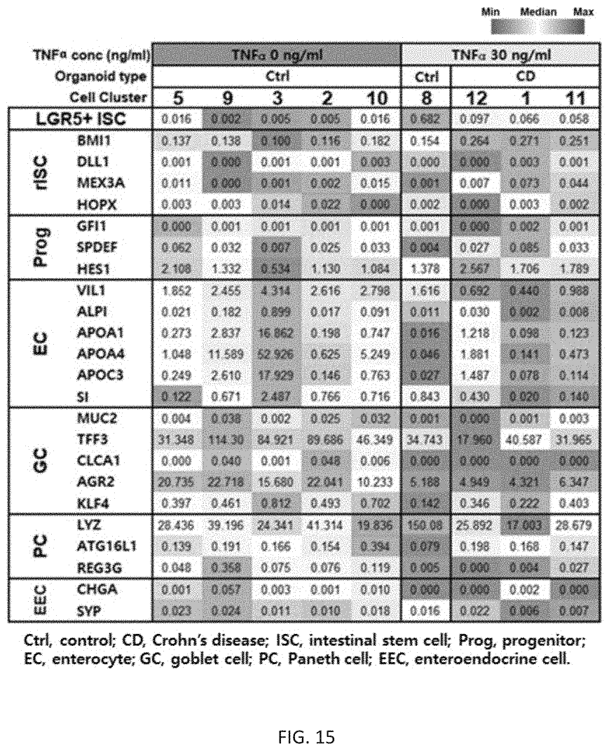

As a result, referring to FIG. 15, it was confirmed that the expression of differentiation cell-related markers increased in the TNFα-untreated organoids, whereas the expression of markers related to stem cells or progenitor cells increased in the TNFα-treated organoids. Interestingly, it was confirmed that, when the organoids were treated with TNFα, LGR5+ expression of cluster 8 increased in the normal person-derived organoids, but was insignificant in the Crohn's disease patient-derived organoids.

These results suggest that the decrease in mucosal regeneration ability that appears in Crohn's disease patients is associated with a decrease in LGR5+ cells. The presumed cause of the decrease may be abnormalities in the function of LGR5+ active intestinal stem cells themselves, but may also be impairment in the process in which reserve intestinal stem cells characterized by the expression of BMI1+, DLL1+, MEX3A, etc. dedifferentiate into LGR5+ stem cells to compensate for apoptosis induced by TNFα. Alternatively, the cause may be that the reduction of Paneth cells constituting the stem cell niches induce the reduction of LGR5+ stem cells.

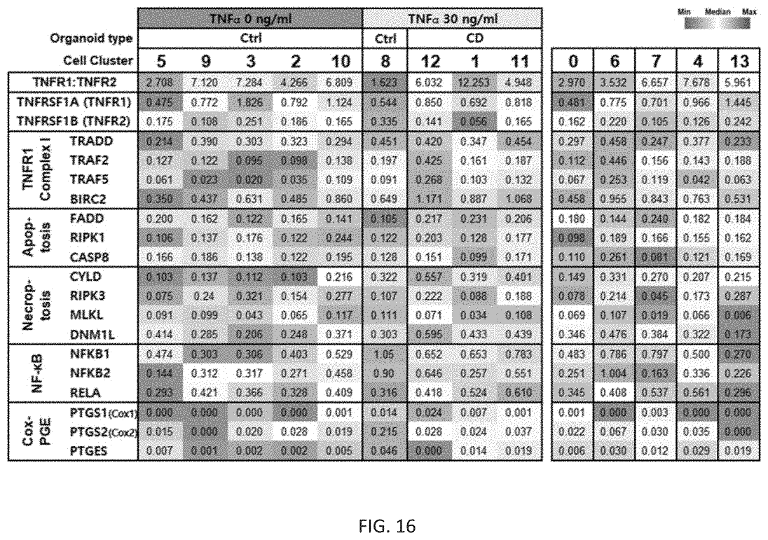

Referring to FIG. 16, in cluster 8 in which LGR5+ cells are present, the expression of TNFSF1B (TNFR2) and NF-κB genes increased, and particularly, the expression of PTGS2 (Cox2) and PTGES increased. Cox2-PTGES is expected to increase the production of PGE2 through the signaling pathway. A previous study reported that PGE2 increases the expression of LGR5 through EP2 receptor and promotes the regeneration and survival of LGR5+ stem cells. Therefore, it can be expected that PGE2 will promote the regeneration and survival of LGR5+ stem cells in intestinal organoids damaged by TNFα, thereby promoting intestinal mucosa regeneration.

### Experimental Example 4. Confirmation of Intestinal Mucosa Regeneration by Combination of PGE2 and Infliximab

In order to compare the changes in the mucosal regeneration ability of normal person- and Crohn's disease patient-derived intestinal organoids by treatment with PGE2 and/or a TNFα inhibitor, organoid reconstitution assay, MTT assay and wound healing assay were performed, and to examine changes in LGR5-expressing cells of intestinal organoids, FACS analysis was performed.

As the TNFα inhibitor, the anti-TNFα antibody infliximab (IFX) was used.

4-1. Effect of Infliximab and PGE2 on Normal Person-Derived Organoids

Whether the changes in normal intestinal epithelial cells by TNFα are restored by infliximab, PGE2 or a combination of infliximab and PGE2 was evaluated using organoid reconstitution assay and MTT assay.

Normal person-derived organoids were cultured in a maintenance medium for 2 days to obtain a stable number of organoids, and then the medium was replaced with a differentiation medium every 2 days. For each experimental group, human recombinant TNFα and PGE2 (Cayman Chemical Company) or infliximab were added to the culture medium every day. The organoid reconstitution rate was calculated by comparing the number of organoids on day 4 and the number of organoids on day 10.

To evaluate cell viability, MTT assay was performed in the same manner as in Experimental Example 2. Statistical analysis was performed by ANOVA using Bonferroni's multiple comparison test (* p<0.05, ** p<0.01 and *** p<0.001).

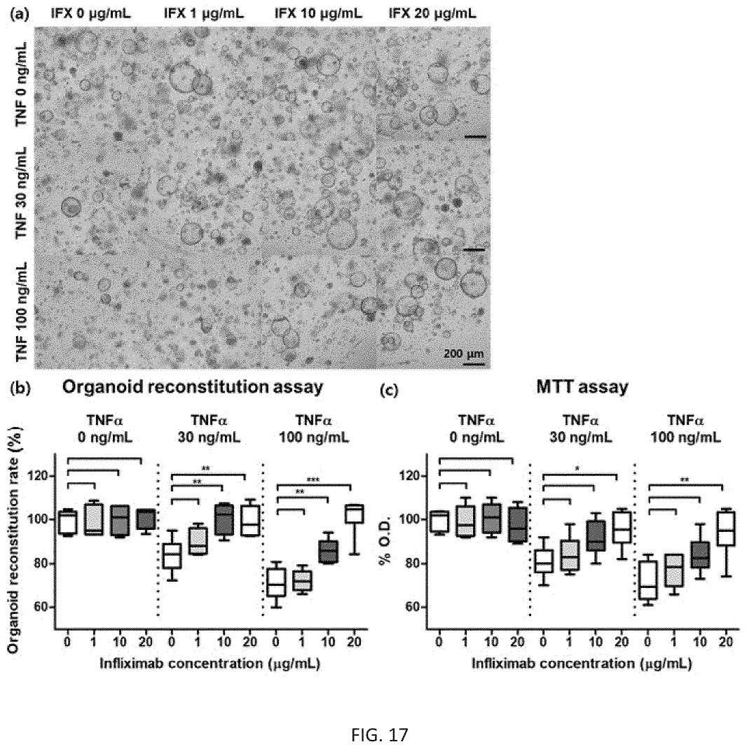

Changes in organoid reconstitution rate and cell viability depending on the concentrations of PGE2 and infliximab were examined. Referring to FIG. 17, normal person-derived organoids were treated with 0, 1, 10, and 20 μg/mL of infliximab while being treated with 0, 30 and 100 ng/mL of TNFα, and organoid reconstitution assay and MTT assay were performed. As a result, in a manner dependent on the concentration of TNFα, the organoid reconstitution rate decreased and the cell viability also decreased. In the normal person-derived intestinal organoids treated with 30 ng/mL of TNFα, as the concentration of infliximab increased, the organoid reconstitution rate and cell viability were restored, and thus the organoid reconstitution rate and cell viability when the organoids were treated with 10 or 20 μg/mL of infliximab together with TNFα were similar to those when the organoids were not treated with TNFα, and were significantly restored compared to when the organoids were not treated with infliximab. Even in the normal person-derived intestinal organoids treated with 100 ng/mL of TNFα, as the concentration of infliximab increased, the organoid reconstitution rate and cell viability were restored, and thus the organoid reconstitution rate and cell viability when the organoids were treated with 20 μg/mL of infliximab together with TNFα were similar to those when the organoids were not treated with TNFα, and were significantly restored compared to when the organoids were not treated with infliximab.

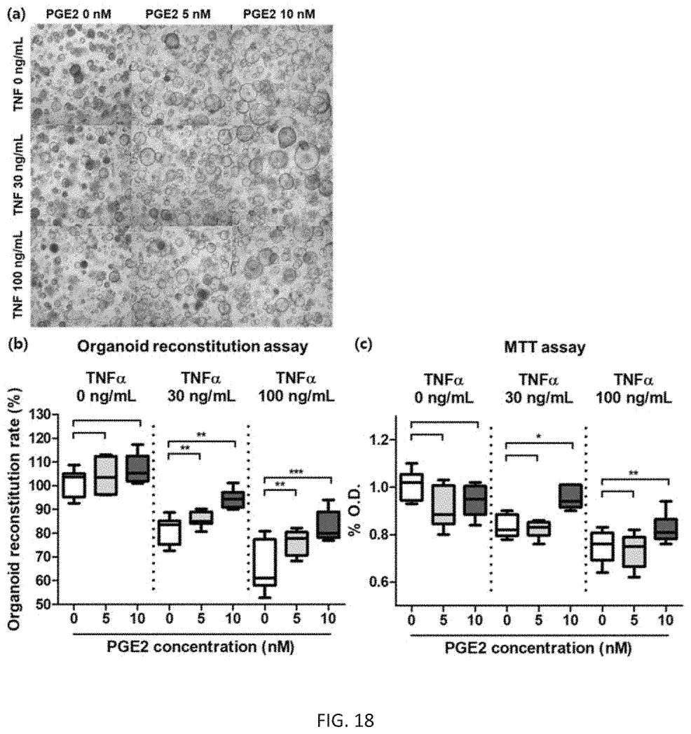

Referring to FIG. 18, normal person-derived organoids were treated with 0, 5 or 10 nM of PGE2 while being treated with 0, 30 or 100 ng/mL of TNFα, and organoid reconstitution assay and MTT analysis were performed. As a result, in the normal person-derived intestinal organoids treated with 30 ng/mL of TNFα, as the concentration of PGE2 increased, the organoid reconstitution rate and cell viability were restored, and thus the organoid reconstitution rate and cell viability when the organoids were treated with 10 nM of PGE2 together with TNFα were similar to those when the organoids were not treated with TNFα, and were significantly restored compared to when the organoids were not treated with PGE2. Even in the normal person-derived intestinal organoids treated with 100 ng/mL of TNFα, as the concentration of PGE2 increased, the organoid reconstitution rate and cell viability were restored, and thus the organoid reconstitution rate and cell viability when the organoids were treated with 10 nM of PGE2 together with TNFα were significantly improved compared to when the organoids were not treated with PGE2.

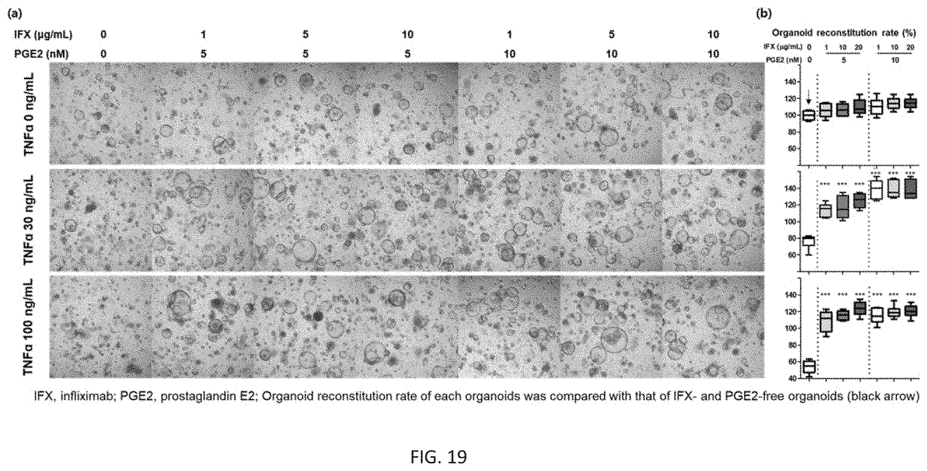

Referring to FIG. 19, the normal person-derived organoids were treated with various concentrations of infliximab (low concentration=1 μg/mL, therapeutic drug concentration=5 μg/mL, high concentration=10 μg/mL) and various concentrations of PGE2 (low concentration=5 nM, high concentration=10 nM) while being treated with 0, 30 and 100 ng/mL of TNFα, and the organoid reconstitution rates were analyzed. As a result, it could be confirmed that, when the organoids were treated with a high concentration of infliximab or PGE2, the restoration of the organoid reconstitution rates was improved as expected, and even when the normal person-derived intestinal organoids treated with or 100 ng/mL of TNFα were treated with a combination of a low concentration of infliximab (1 μg/mL) and a low concentration of PGE2 (5 nM), the reconstitution rate of the normal person-derived organoids was restored.

4-2. Effects of Infliximab and PGE2 on Crohn's Disease Patient-Derived Organoids

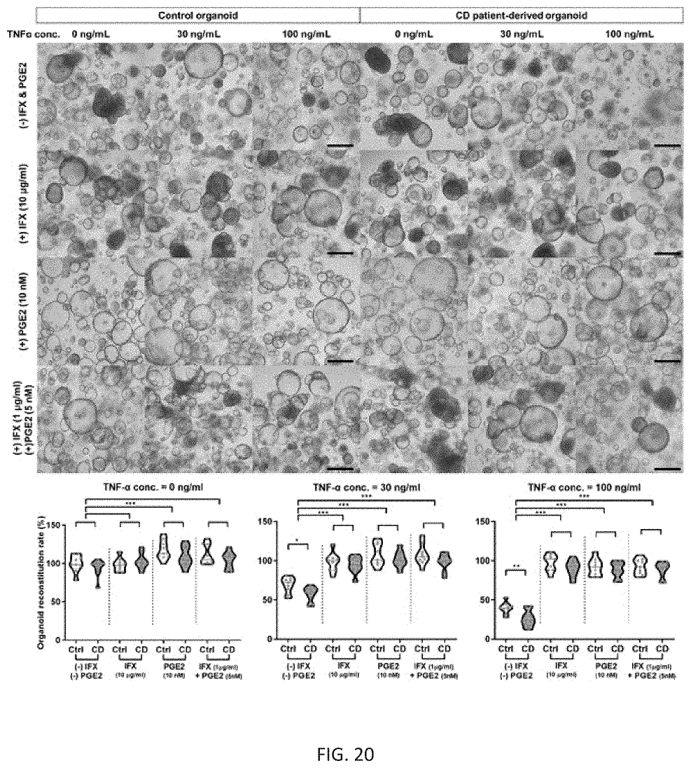

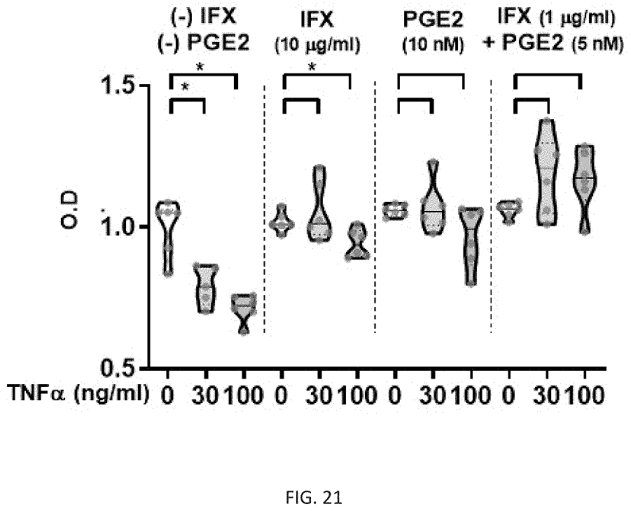

FIGS. 20 and 21 show the results of analyzing the changes in organoid reconstitution rate and cell viability of TNFα-treated Crohn's disease patient-derived organoids by infliximab and PGE2. Cases in need of actual infliximab treatment or potential PGE2 treatment are Crohn's disease patients, and based on the information obtained from the normal person-derived organoids, the effects of infliximab and PGE2 on Crohn's disease patient-derived organoids with reduced intestinal epithelial regeneration ability were evaluated.

When the Crohn's disease patient-derived intestinal organoids were treated with 30 or 100 ng/mL of human recombinant TNFα, the reconstitution rate of the Crohn's disease patient-derived intestinal organoids decreased in a concentration-dependent manner and apoptosis increased in a concentration-dependent manner. Treatment with a high concentration of infliximab (10 μg/mL) and a high concentration of PGE2 (10 nM) restored the reconstitution rate and cell viability of the Crohn's disease patient-derived organoids, treated with 30 or 100 ng/mL of TNFα, to levels similar to those of TNFα-untreated normal person-derived organoids. It was confirmed that, when the Crohn's disease patient-derived intestinal organoids were treated with a low concentration of infliximab (1 μg/mL) and a low concentration of PGE2 (5 nM) together with TNFα, the reconstitution rate and cell viability of the organoids were maintained at levels obtained when the organoids were treated with a high concentration of infliximab or PGE2.

In addition, wound healing assay was performed in the same manner as in Experimental Example 7.

For the Crohn's disease patient-derived organoids of each experimental group, 30 ng/mL of human recombinant TNFα, or 10 nM of PGE2, and 1 or 10 μg/mL of infliximab were added to the culture medium daily. Statistical analysis was performed by ANOVA using Bonferroni's multiple comparison test (** p<0.01 and *** p<0.001).

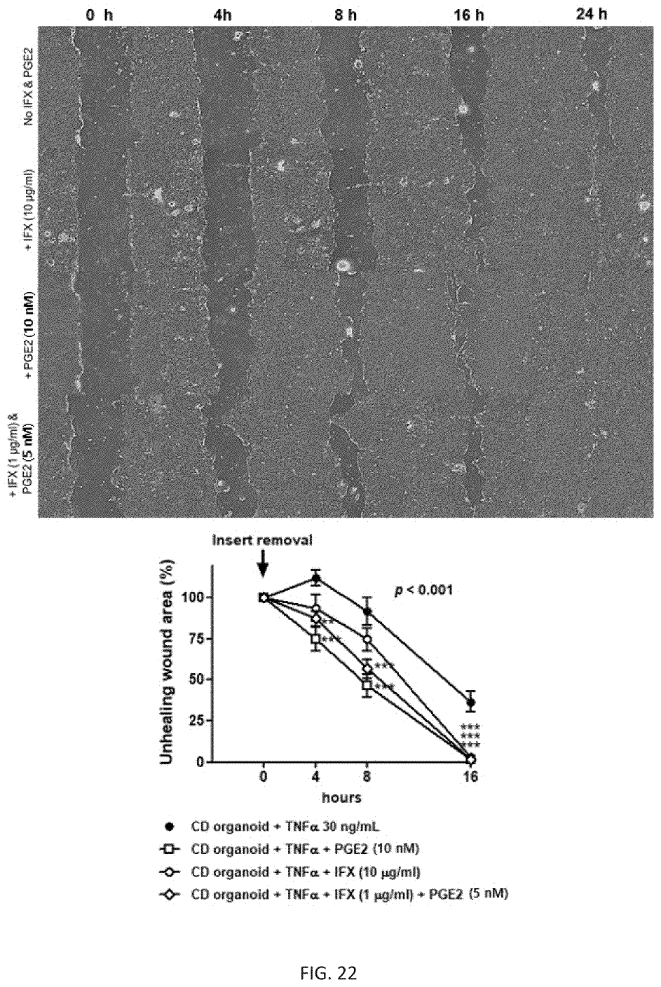

As a result, referring to FIG. 22, it was confirmed that, when the organoids were treated with a high concentration of infliximab or PGE2 together with TNFα, the wound area was recovered after 16 hours, and thus the unhealed wound area was reduced, compared to when the organoids were treated with TNFα alone. In addition, it was confirmed that, when the organoids were treated with a low concentration of infliximab or PGE2 together with TNFα, the wound area was recovered after 16 hours, and thus the unhealed wound area was reduced, similar to when the organoids were treated with a high concentration of infliximab or PGE2. Therefore, it could be confirmed that the combination of a low concentration of infliximab and a low concentration of PGE2 improved the wound healing ability reduced by TNFα.

These results suggest that PGE2 and the TNFα inhibitor can improve the intestinal mucosal healing ability of Crohn's disease patient-derived organoids having reduced intestinal epithelial regeneration ability induced by TNFα. Thus, the combination of a low concentration of a TNFα inhibitor and a low concentration of PGE2 can be expected to be useful in improving the therapeutic effect against Crohn's disease.

4-3. Real-Time Quantitative Reverse Transcription-PCR (qPCR)

For the Crohn's disease patient-derived organoids of each experimental group, 0, 30 or 100 ng/mL of human recombinant TNFα, 0, 5 or 10 nM of PGE2, and 0, 1 or 10 μg/mL of infliximab were added to the culture medium daily.

Total RNA was extracted from intestinal organoids using the RNeasy Mini Kit (QIAGEN). One-step qPCR was performed using the following primers together with One Step PrimeScript™ III RT-qPCR Mix (Takara): LGR5 primers (forward: 5′-aactttggcattgtggaagg-3′, reverse: 5′-acacattgggggtaggaaca-3′). Statistical analysis was performed by ANOVA using Bonferroni's multiple comparison test (* p<0.05 and *** p<0.001).

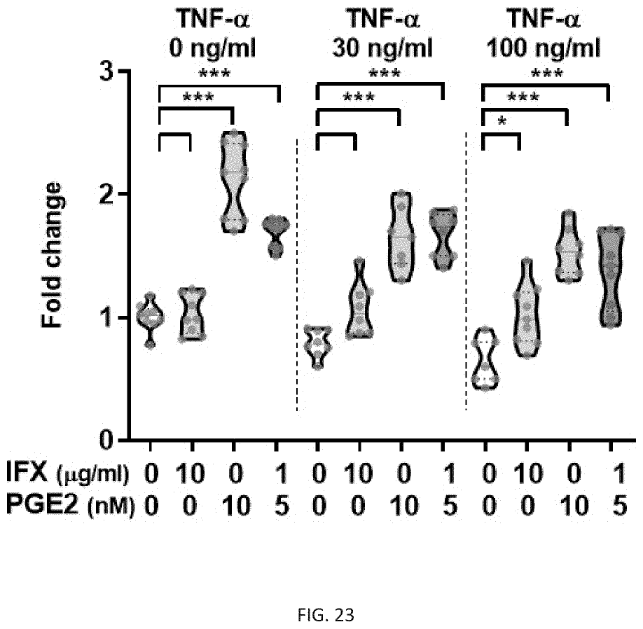

As a result, referring to FIG. 23, it was confirmed that, when the organoids were treated with a high concentration of infliximab together with TNFα, there was no difference in LGR5 expression depending on the concentration of TNFα, whereas when the organoids were treated with a high concentration of PGE2 together with TNFα, LGR5 expression increased.

In addition, it was confirmed that, when the organoids were treated with low concentrations of infliximab and PGE2 together with TNFα, LGR5 expression increased similar to when the organoids were treated with a high concentration of PGE2.

4-4. FACS Analysis

For the Crohn's disease patient-derived organoids of each experimental group, 30 ng/mL of human recombinant TNFα, or 10 nM of PGE2, and 1 or 10 μg/mL of infliximab were added to the culture medium daily.

To prepare single-cell suspensions from organoids, organoids were incubated with TrypLE Express in a water bath at 37° C. for 30 minutes. Then, the cells were washed with basal medium and collected by filtration through a 40-μm cell strainer. Flow cytometry for detecting the marker was performed using 1×10⁵ cells. Cells were incubated with human Lgr5/GPR49 antibody (R&D Systems) in FACS buffer for 30 min. Unbound antibodies were washed out, and the cells were incubated with Alexa Fluor 488-conjugated anti-mouse immunoglobulin G secondary antibody (Thermo Fisher) for 30 minutes. The cells were then washed, resuspended in FACS buffer, and analyzed on the FACS Aria III instrument (BD Biosciences) to evaluate LGR5+ ISCs population.

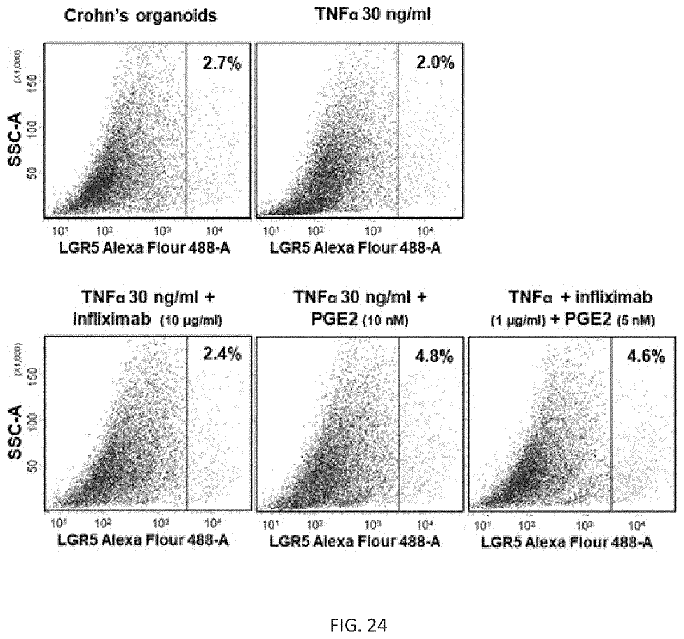

As a result, referring to FIG. 24, it was confirmed that, when the organoids were treated with a high concentration of infliximab together with TNFα, there was no difference in LGR5 expression from when the organoids were treated with TNFα, whereas when the organoids were treated with a high concentration of PGE2 together with TNFα, LGR5 expression increased. In addition, it was confirmed that, when the organoids were treated with low concentrations of infliximab and PGE2 together with TNFα, LGR5 expression increased similar to when the organoids were treated with a high concentration of PGE2.

Taking the results together, a high concentration of PGE2 was able to improve intestinal mucosa formation and mucosal healing abilities by inducing proliferation of LGR5-expressing cells in the intestines of Crohn's disease patients. When a low concentration of the TNFα inhibitor and PGE2 were used in combination, the adverse effect on mucosal healing, which occurs because the low concentration of the TNFα inhibitor does not completely block the effect of TNFα, could be eliminated by inducing the proliferation of LGR5-expressing intestinal stem cells by PGE2, thereby increasing intestinal mucosa formation and mucosal healing abilities to normal levels.

Since the TNFα inhibitor is expensive, it is required to reduce the amount of TNFα inhibitor used, in order to reduce medical costs. In addition, there are many cases where it is often not possible to maintain an appropriate therapeutic drug concentration of the TNFα inhibitor in the body, due to the generation of anti-TNFα inhibitor antibodies, etc. That is, in the case in which the amount of TNFα inhibitor used has to be reduced and the therapeutic concentration of the TNFα inhibitor cannot be maintained, a combination of PGE2 and the TNFα inhibitor can be effectively used to for the treatment of Crohn's disease.

So far, the present invention has been described with reference to the preferred embodiments. Those of ordinary skill in the art to which the present invention pertains will appreciate that the present invention may be embodied in modified forms without departing from the essential characteristics of the present invention. Therefore, the disclosed embodiments should be considered from an illustrative point of view, not from a restrictive point of view. The scope of the present invention is defined by the claims rather than the foregoing description, and all differences within the scope equivalent thereto should be construed as being included in the present invention.

1. A pharmaceutical composition for preventing or treating inflammatory bowel disease containing a tumor necrosis factor alpha inhibitor and prostaglandin E2 as active ingredients.

2. The pharmaceutical composition of claim 1, wherein the tumor necrosis factor alpha inhibitor is at least one selected from the group consisting of an antibody, a fusion protein, and a compound, which specifically bind to tumor necrosis factor alpha.

3. The pharmaceutical composition of claim 1, wherein the antibody is at least one selected from the group consisting of infliximab, adalimumab, certolizumab pegol, golimumab, and etanercept.

4. The pharmaceutical composition of claim 1, which improves reconstitution of damaged intestinal mucosa and intestinal mucosal healing ability.

5. The pharmaceutical composition of claim 1, which induces proliferation of LGR5-expressing intestinal stem cells.

6. (canceled)
